(19) United States
(12) Patent Application Publication
(10) Pub. No.: US 2023/0402152 A1
(43) Pub. Date: Dec. 14, 2023

(54) DOSE-DIRECTED RADIATION THERAPY PLAN GENERATION USING COMPUTER MODELING TECHNIQUES

(71) Applicant: VARIAN MEDICAL SYSTEMS, INC., Palo Alto, CA (US)

(72) Inventors: Simeng Zhu, Palo Alto, CA (US); Alexander E. Maslowski, Palo Alto, CA (US); Esa Kuusela, Espoo (FI)

(73) Assignee: VARIAN MEDICAL SYSTEMS, INC., Palo Alto, CA (US)

(21) Appl. No.: 17/839,206

(22) Filed: Jun. 13, 2022

(51) Int. Cl. G16H 20/40; G06N 20/00

(52) U.S. Cl. CPC G16H 20/40; G06N 20/00

(57) ABSTRACT

Provided herein are methods and systems to train and execute a computer model that uses artificial intelligence methodologies (e.g., deep learning) to learn and predict Multi-leaf Collimator (MLC) openings and control weights for a radiation therapy treatment plan.

### TECHNICAL FIELD

This application relates generally to using data analysis techniques to model and predict attributes for radiation therapy treatment and to control a radiation therapy machine.

### BACKGROUND

Radiation therapy treatment planning (RTTP) is a complex process that contains specific guidelines, protocols, and instructions adopted by different medical professionals, such as the clinicians, the medical device manufacturers, and the like. Typically, identifying and applying guidelines to implement radiation therapy treatment are performed by complex computer models that receive treatment objectives from a treating physician and identify suitable attributes of the RTTP. For instance, the treating physicians may identify the treatment modality (e.g., choose between the volumetric modulated arc therapy (VMAT) or intensity-modulated radiation therapy (IMRT)). The treating physician may then input various objectives and goals to be achieved via the treatment, such as dose objectives to be achieved for one or more structures of the patient. A software solution may then use various methods to calculate attributes of the patient's treatment, such as determining beam limiting device angles and radiation emitting attributes. In the case of IMRT, the beam delivery directions and number of beams are the specifically relevant variables that must be decided, whereas, for VMAT, the software solution may need to choose the number of arcs and their corresponding start and stop angles.

Therefore, for plan generation—and for VMAT plans, in particular—some software solutions use an iterative, trial-and-error process to optimize various attributes of the RTTP. For instance, after the treating physician inputs treatment objectives, the software solution iteratively analyzes different possibilities (e.g., different iterations of different attributes within a large search space) to identify which iteration yields the best (or acceptable) results. Specifically, a VMAT plan optimizer software solution may configure a set of linear accelerator machine instructions, such as Multi-leaf Collimator (MLC) sequence and control point weights to deliver a dose that satisfies the treatment objectives reflecting a radiation oncologist's goals. As a result, the software solutions may require substantial computing resources and may not produce timely results.

### SUMMARY

For the aforementioned reasons, there is a desire for a system that can rapidly and accurately analyze plan/treatment objectives and patient information to provide RTTP attributes. Using the methods and systems discussed herein, a computer model (e.g., artificial intelligence (AI) or machine learning (ML) model) can leverage various AI/ML techniques, such as deep learning, to propose a set of VMAT machine instructions that include MLC sequence and control point weights, given the objectives of desired dose distribution and associated planning structures.

Using the methods and systems discussed herein, a processor can use deep learning to train a model to predict MLC openings for a patient's treatment. Specifically, the computer model may use a data-driven, statistical learning method (e.g., deep learning) to build the correlations between image-level features of dose distribution and linear accelerator machine instructions, such as MLC sequence and control point weights. With these learned correlations, the computer model (e.g., software solution that generates the RTTP) can rapidly generate a set of machine instruction that allow the delivery of a certain desired dose distribution.

Currently, VMAT plans are usually generated in the clinic with the use of plan optimizers alone. However, this process is time-consuming because multiple iterations of optimizations are usually required (partly due to reliance on DVH-based two-dimensional (2D) planning objectives) and each iteration of optimization taking a long time to analyze due to the large search space. Using the methods and systems described herein, a computer model may reduce the time it would take a conventional software solution to generate an RTTP. The described computer model may reduce the time by improving current solutions in at least two different ways. First, the methods and systems described herein may use three-dimensional (3D) dose distributions as the planning objective rather than the 2D DVH-based planning objectives. Second, the methods and systems described herein utilize deep learning and/or other AI/ML techniques to rapidly predict a set of linear accelerator machine instruction, which could produce the desired dose distribution with minimal need for an optimizer.

Using the methods and systems described therein, the server may train a model using various AI/ML techniques such as deep learning, to generate a full set of linear accelerator machine instructions to deliver a VMAT plan (including MLC openings and control point weights) using desired 3D dose distribution, radiation therapy planning structures, and simulation medical images (e.g., computerized tomography (CT) images) as inputs.

The model may predict the shape of MLC openings and the corresponding weight for each control point. “Weights,” as used herein may refer to a movement attribute of the linear accelerator at a particular point (control points) during its rotation. For instance, the weight may indicate a velocity (or an angular velocity) of movement of the linear accelerator at a control point. In another example, the weight may indicate an angle of movement. In some embodiments, the weight may correspond to multiple attributes. For instance, the weight may correspond to a movement attribute and dosage (e.g., indicating how the accelerator is moving and the dosage emitted at the same time). Essentially, the control point weight may indicate how radiation is administered at a particular location/angle.

MLC openings and control point weights may change during the treatment. For instance, each position of the linear accelerator may correspond to a particular control point weight and MLC opening. In a non-limiting example, an MLC may have a first opening and a first control point weight at the first control point and a completely different MLC opening and control point weight at a second control point. Using the methods and systems discussed herein, a server can predict desired MLC openings and control point weights at any given time or location of the linear accelerator that would yield results satisfying the plan objectives.

In an embodiment, a method may comprise receiving, by a processor, treatment objectives for a patient including at least a dose-volume for at least one structure of the patient to be treated via a radiation therapy machine; executing, by the processor, an artificial intelligence model to predict an attribute of a Multi-Leaf Collimator (MLC) opening and a corresponding movement attribute of an accelerator of the radiation therapy machine, wherein the artificial intelligence model is trained via a training dataset that comprises training treatment objectives and attributes associated with previously performed radiation therapy treatments comprising at least actual or projected dose-volume histograms for the treated patients and corresponding MLC opening positions; and presenting, by the processor, a predicted MLC opening position and a corresponding predicted movement attribute of the accelerator of the radiation therapy machine.

The artificial intelligence model may be trained via a deep learning protocol to correlate the actual or projected dose-volume histogram for the treated patients and corresponding MLC opening positions.

The predicted MLC opening position may be a binary mask indicating opening of the MLC.

The predicted movement attribute may be a time associated with the accelerator's movement.

The predicted movement attribute may be an angle associated with the accelerator's movement.

The artificial intelligence model may further predict a sequence of MLC openings for the patient.

The artificial intelligence model may utilize a loss function in accordance with MLC opening restrictions.

The method may further comprise generating, by the processor, machine-readable instructions in accordance with the predicted MLC openings and corresponding predicted movement attributes.

The method may further comprise transmitting, by the processor, the machine-readable instructions to the radiation therapy machine.

In another embodiment, a computer system may comprise a server comprising a processor and a non-transitory computer-readable medium containing instructions that when executed by the processor causes the processor to perform operations comprising: receiving treatment objectives for a patient including at least a dose-volume for at least one structure of the patient to be treated via a radiation therapy machine; executing an artificial intelligence model to predict an attribute of a Multi-Leaf Collimator (MLC) opening and a corresponding movement attribute of an accelerator of the radiation therapy machine, wherein the artificial intelligence model is trained via a training dataset that comprises training treatment objectives and attributes associated with previously performed radiation therapy treatments comprising at least actual or projected dose-volume histograms for the treated patients and corresponding MLC opening positions; and presenting a predicted MLC opening position and a corresponding predicted movement attribute of the accelerator of the radiation therapy machine.

The artificial intelligence model may be trained via a deep learning protocol to correlate the actual or projected dose-volume histogram for the treated patients and corresponding MLC opening positions.

The predicted MLC opening position may be a binary mask indicating opening of the MLC.

The predicted movement attribute may be a time associated with the accelerator's movement.

The predicted movement attribute may be an angle associated with the accelerator's movement.

The artificial intelligence model may further predict a sequence of MLC openings for the patient.

The artificial intelligence model may utilize a loss function in accordance with MLC opening restrictions.

The instructions may further cause the processor to generate machine-readable instructions in accordance with the predicted MLC openings and corresponding predicted movement attributes.

The instructions may further cause the processor to transmit the machine-readable instructions to the radiation therapy machine.

In another embodiment, a system may comprise a server having one or more processors configured to receive treatment objectives for a patient including at least a dose-volume for at least one structure of the patient to be treated via a radiation therapy machine; execute an artificial intelligence model to predict an attribute of a Multi-Leaf Collimator (MLC) opening and a corresponding movement attribute of an accelerator of the radiation therapy machine, wherein the artificial intelligence model is trained via a training dataset that comprises training treatment objectives and attributes associated with previously performed radiation therapy treatments comprising at least actual or projected dose-volume histograms for the treated patients and corresponding MLC opening positions; and present a predicted MLC opening position and a corresponding predicted movement attribute of the accelerator of the radiation therapy machine.

The artificial intelligence model may be trained via a deep learning protocol to correlate the actual or projected dose-volume histogram for the treated patients and corresponding MLC opening positions.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of an artificial intelligence (AI) plan generation system, according to an embodiment.

FIG. 2A illustrates a process flow diagram executed in an AI plan generation system, according to an embodiment.

FIG. 2B illustrates a process flow diagram executed in an AI model in an AI plan generation system, according to an embodiment.

FIG. 2C illustrates a process flow diagram executed in an AI model in an AI plan generation system, according to an embodiment.

FIG. 3 illustrates a visual representation of processing data to train an AI model in an AI plan generation system, in accordance with an embodiment.

FIG. 4 illustrates a visual representation of processing data to train an AI model in an AI plan generation system, in accordance with an embodiment.

FIG. 5 illustrates a visual representation of training an AI model in an AI plan generation system, in accordance with an embodiment.

FIG. 6 illustrates a non-limiting example of data presented in an AI plan generation system, in accordance with an embodiment.

FIG. 7 illustrates a non-limiting example of data presented in an AI plan generation system, in accordance with an embodiment.

FIG. 8 illustrates a process flow diagram executed in an AI plan generation system, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

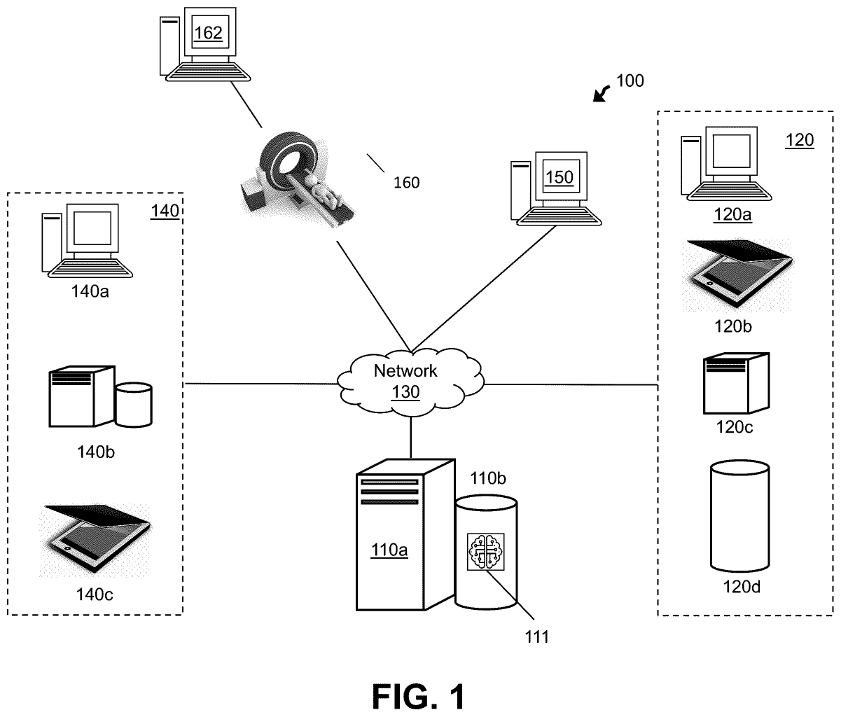

FIG. 1 illustrates components of a system 100 for an artificial intelligence plan generation system, according to an embodiment. The system 100 may include an analytics server 110a, system database 110b, an AI model 111, electronic data sources 120a-d (collectively electronic data sources 120), end-user devices 140a-c (collectively end-user devices 140), an administrator computing device 150, a medical device 160, and a medical device computer 162. Various components depicted in FIG. 1 may belong to a radiation therapy treatment clinic at which patients may receive radiation therapy treatment, in some cases via one or more radiation therapy machines (e.g., medical device 160).

The system 100 is not confined to the components described herein and may include additional or other components, not shown for brevity, which are to be considered within the scope of the embodiments described herein.

The above-mentioned components may be connected to each other through a network 130. Examples of the network 130 may include, but are not limited to, private or public local-area networks (LAN), wireless local-area networks (WLAN), metropolitan-area networks (MAN), wide-area networks (WAN), and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), EDGE (Enhanced Data for Global Evolution) network.

The analytics server 110a may generate and display an electronic platform configured to use various AI models 111 (including artificial intelligence and/or machine learning models) for receiving patient information and outputting the results of execution of the AI models 111. The electronic platform may include graphical user interfaces (GUI) displayed on each electronic data source 120, the end-user devices 140, the medical device 160, and/or the administrator computing device 150. An example of the electronic platform generated and hosted by the analytics server 110a may be a web-based application or a website configured to be displayed on different electronic devices, such as mobile devices, tablets, personal computers, and the like.

The information displayed by the electronic platform can include, for example, input elements to receive data associated with a patient to be treated (e.g., plan objectives) and display results of predictions produced by the AI model 111 (e.g., predicted MLC opening image or numerical sequence data and/or control point weights). The analytics server 110a may then display the results for a medical professional and/or directly revise one or more operational attributes of the medical device 160. In some embodiments, the medical device 160 can be a diagnostic imaging devices or a treatment delivery device.

The analytics server 110a may be any computing device comprising a processor and non-transitory machine-readable storage capable of executing the various tasks and processes described herein. The analytics server 110a may employ various processors such as central processing units (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

The electronic data sources 120 may represent various electronic data sources that contain, retrieve, and/or access data associated with a medical device 160, such as operational information associated with previously performed radiation therapy treatments (e.g., electronic log files or electronic configuration files), data associated with previously monitored patients (e.g., RTTPs of previous patients and their corresponding treatment attributes and other machine instructions included within a radiotherapy treatment file) or participants in a study to train the AI models 111 discussed herein. For instance, the analytics server 110a may use the clinic computer 120a, medical professional device 120b, server 120c (associated with a physician and/or clinic), and database 120d (associated with the physician and/or the clinic) to retrieve/receive data associated with the medical device 160. The analytics server 110a may retrieve the data from the end-user devices 120, generate a training dataset, and train the AI models 111. The analytics server 110a may execute various algorithms to translate raw data received/retrieved from the electronic data sources 120 into machine-readable objects that can be stored and processed by other analytical processes as described herein.

End-user devices 140 may be any computing device comprising a processor and a non-transitory machine-readable storage medium capable of performing the various tasks and processes described herein. Non-limiting examples of an end-user device 140 may be a workstation computer, laptop computer, tablet computer, and server computer. In operation, various users may use end-user devices 140 to access the GUI operationally managed by the analytics server 110a. Specifically, the end-user devices 140 may include clinic computer 140a, clinic server 140b, and a medical processional device 140c. Even though referred to herein as “end-user” devices, these devices may not always be operated by end-users. For instance, the clinic server 140b may not be directly used by an end user. However, the results stored onto the clinic server 140b may be used to populate various GUIs accessed by an end user via the medical professional device 140c.

The administrator computing device 150 may represent a computing device operated by a system administrator. The administrator computing device 150 may be configured to display radiation therapy treatment attributes generated by the analytics server 110a (e.g., various analytic metrics determined during training of one or more machine learning models and/or systems); monitor various models 111 utilized by the analytics server 110a, electronic data sources 120, and/or end-user devices 140; review feedback from end-user devices 140; and/or facilitate training or retraining (calibration) of the AI model 111 that are maintained by the analytics server 110a.

The medical device 160 may be a radiation therapy machine configured to implement a patient's radiation therapy treatment. The medical device 160 may include a linear accelerator and MLC configured to control the emission of radiation to a patient. The medical device 160 may also be in communication with a medical device computer 162 that is configured to display various GUIs discussed herein. For instance, the analytics server 110a may display the results predicted by the AI model 111 onto the computing devices described herein. In a non-limiting example, the GUI may display the projected MLC opening or control point weights that were predicted by the AI models 111.

The AI model 111 may be stored in the system database 110b. The AI model 111 may be trained using data received/retrieved from the electronic data sources 120 and may be executed using data received from the end-user devices, the medical device 160, and/or the sensor 163. In some embodiments, the AI model 111 may reside within a data repository local or specific to a clinic. In various embodiments, the AI models 111 use one or more deep learning engines to generate MLC openings and control weights for a patient.

It should be understood that any alternative and/or additional machine learning model(s) may be used to implement similar learning engines. The deep learning engines can include processing pathways that are trained during a training phase. Once trained, deep learning engines may be executed (e.g., by the analytics server 110a) to generate predicted treatment attributes.

As described herein, the analytics server 110a may store the AI model 111 (e.g., neural networks, random forest, support vector machines, regression models, recurrent models, etc.) in an accessible data repository. The analytics server 110a may retrieve the AI models 111 and train the AI models 111 to predict treatment attributes for a patient including MLC openings and control point weights.

Various machine learning techniques may involve “training” the machine learning models to predict treatment attributes, including supervised learning techniques, unsupervised learning techniques, or semi-supervised learning techniques, among others. In a non-limiting example, the predicted patient attribute may indicate an MLC opening and/or control point weights. The AI model 111 can therefore be used to predict a real-time MLC opening and control point weights (e.g., location and orientation of the linear accelerator).

In practice, the data used for the training dataset may be user-generated through observations and experience to facilitate training the AI models 111. For example, training data may be received and monitored during previous radiation therapy treatments provided for prior patients. In another example, the training data may be a dataset that includes treatment objectives (e.g., DVH objectives), RTTPs, projected dose distribution, MLC openings, and control weights associated with previously treated patients. Training data may be processed via any suitable data augmentation approach (e.g., normalization, encoding, or any combination thereof) to produce a new dataset with modified properties to improve the quality of the data. The methods and systems described herein are not limited to training AI models based on patients who have been previously treated. For instance, the training dataset may include data associated with any set of participants (not patients) who are willing to be monitored for the purposes of generating the training dataset.

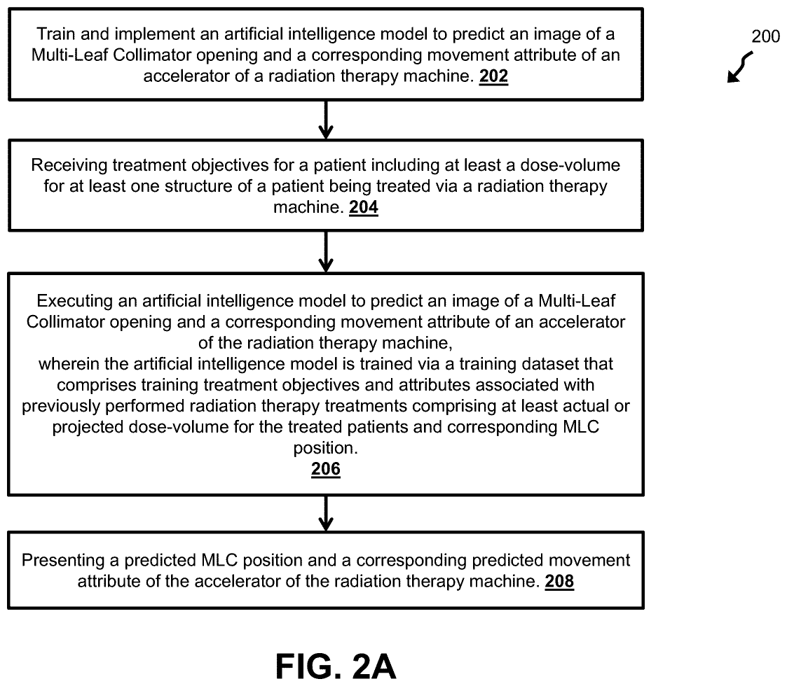

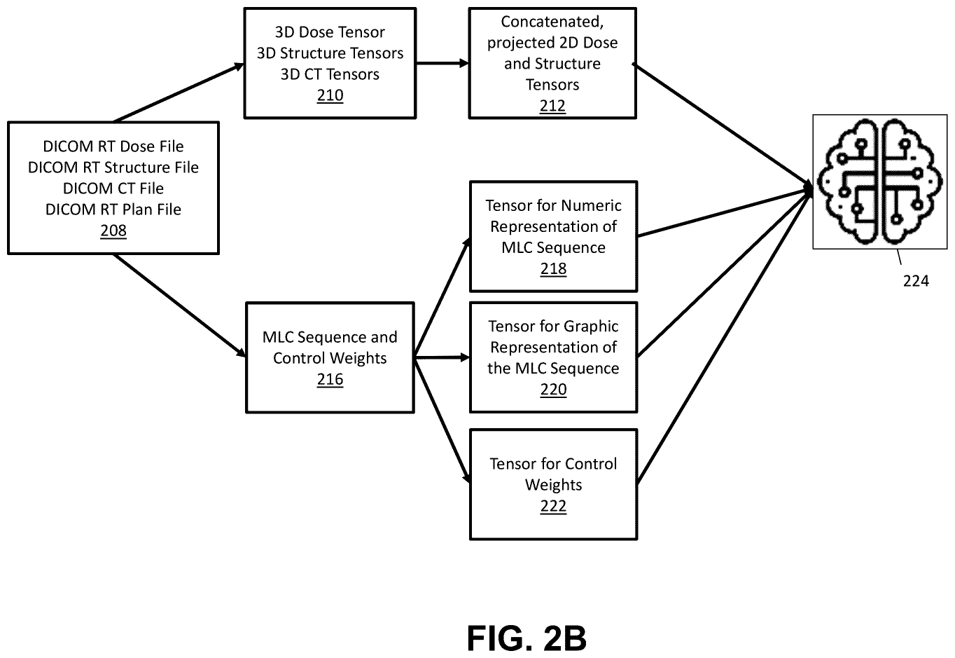

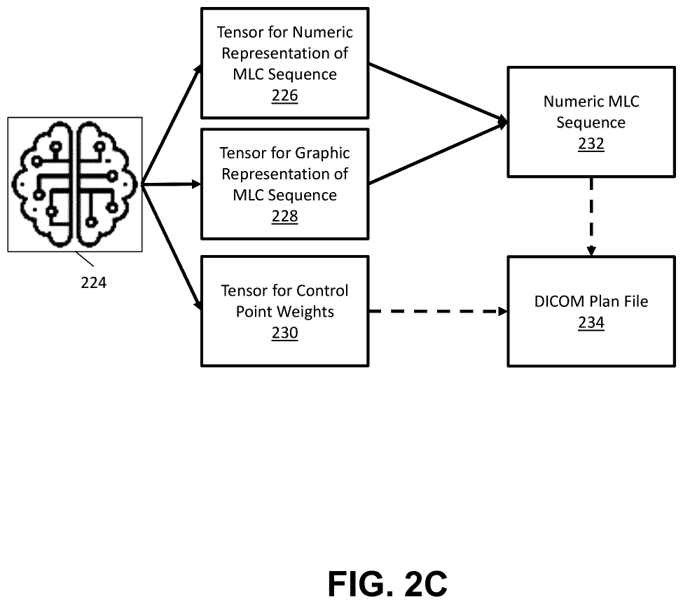

Referring to FIG. 2A, a method 200 shows an operational workflow executed in an artificial intelligence plan generation system, in accordance with an embodiment. The method 200 may include steps 202-208. However, other embodiments may include additional or alternative steps or may omit one or more steps altogether. The method 200 is described as being executed by a server, such as the analytics server described in FIG. 1. However, one or more steps of the method 200 may be executed by any number of computing devices operating in the distributed computing system described in FIG. 1. For instance, one or more computing devices may locally perform part or all of the steps described in FIG. 2A.

Using the method 200, an AI model can be trained in accordance with a training dataset to receive required or predicted/projected dose distribution data, such as dose objectives or a projected 3D dose volume, and to predict corresponding MLC openings and movement attributes for a linear accelerator. At implementation, using the method 200, the AI model may be executed in conjunction with a plan optimizer model to prepare a sequence of control weights and MLC openings. The results predicted by the AI model (using the method 200) may be displayed to a clinician for their approval and/or ingested by the radiation therapy machine itself.

At step 202, the analytics server may train an artificial intelligence model to predict an image of a Multi-Leaf Collimator (MLC) opening and a corresponding movement attribute of an accelerator of a radiation therapy machine. Before executing the AI model (e.g., allowing clinicians to use the AI model), the analytics server may first train the AI model and ensure its accuracy. Training the AI model may be accomplished in different stages. For instance, the analytics server may first prepare a training dataset and train the AI model, as described herein and depicted in FIGS. 2B, and 3-5. The analytics server may then implement the AI model, as described herein and depicted in FIG. 2C.

Preparing Training Dataset

The analytics server may train the AI model using a training dataset comprising at least two sets of data associated with a set of previously treated patients (or participants in a clinical trial). First, the training dataset may include RTTP data associated with each patient within the set of patients. For instance, the training data may include various objectives (e.g., dose objectives) inputted by the clinicians. The training data may also include various data associated with how the RTTP was generated. For instance, the training data may include how a plan optimizer (or any other model or software solution) used the plan objectives to prepare an RTTP for the patient. For instance, the training dataset may include projected 3D dose volume. In some embodiments, images projected by the plan optimizer may be obtained and included in the training dataset.

Second, the training dataset may include linear accelerator machine instructions that indicate details associated with how the RTTP was implemented. Linear accelerator machine instructions may refer to how radiation was emitted and how the radiation (having attributes that were generated for the patient by the plan optimizer) was administered to the patient. Specifically, the linear accelerator machine instructions may include a log of instructions received by a radiation therapy machine causing the linear accelerator to adjust and configure the MLC in certain ways (e.g., speed of movement, angle of movement, and/or MLC opening attributes). Accordingly, the training dataset may include MLC openings and their corresponding control weights. Specifically, the training dataset may include a set of MLC opening data and their corresponding time, such that the AI model may recreate a sequence of how the MLC was opened/configured throughout the patient's treatment. The training dataset may also include control weights associated with the linear accelerator throughout the patient's treatment. The linear accelerator machine instructions may be periodically obtained while the patients are to be treated (e.g., via a radiation therapy machine log).

In an embodiment, the training dataset may also include medical images (e.g., CT or 4DCT) depicting the patient's internal organs and RTTP implementation data. The medical images may be actual or predicted by another model. For instance, the training dataset may include a simulation CT image. The information from the simulation CT images may be used to form the basis for the input to the AI model. Specifically, the simulation CT image may allow for the delineation of planning structures, where these structures may allow the generation of an expected 3D dose distribution. This 3D dose distribution (more accurately, its 2D projection images) may be used as the input to the AI model.

The training dataset may also include additional data associated with the patients. For instance, the AI model may consider each patient's demographic information and/or other biological markers (e.g., age, weight, or BMI). As a result, the model may also consider the patient's attributes when considering how the RTTP was implemented. During (and a result of) the treatment, some patients may have physical changes (e.g., weight loss). Therefore, their projected data may change slightly.

When reviewed in totality, the training dataset may include information that could indicate how each patient's RTTP was administered to the patient. Specifically, the training dataset may indicate attributes of the RTTP and corresponding linear accelerator machine instructions (specifically, MLC opening and control weights) for each patient. Each MLC opening and/or control weight can be analyzed in view of its timestamp and a corresponding attribute of RTTP for the patient. Using this data, the AI model may build correlations between linear accelerator machine instructions and image-level features of dose distribution.

The analytics server may then aggregate various data points associated with the set of patients and their treatment to generate an aggregated training dataset. The analytics server may also perform various data cleaning protocols, such as de-duplicating and other analytical protocols to ensure that the training dataset can be ingested by the AI model to produce results.

As depicted in FIG. 2B, before training the AI model, the analytics server may also prepare the data within the training dataset. First, the analytics server may analyze treatment data associated with each patient. Specifically, the analytics server may extract and analyze Imaging and Communications in Medicine (DICOM) radiation therapy files for dose, structure, and CT to construct a 3D dose tensor, 3D structure tensor (e.g., one for each structure), and/or a 3D CT tensor for each patient treated. DICOM, as used herein, may refer to standardized diagnostic imaging that may include DICOM-RT objects (e.g., RT Image, RT Structure Set, RT Plan, RT Dose, RT Beams Treatment Record, RT Brachy Treatment Record, and RT Treatment Summary Record). Even though aspects of the present disclosure discussed DICOM as a standard, it is understood that other standardized or structured data can be used. A tensor, as used herein, may refer to a mathematical element, such as a vector or an array of components, describing functions relevant to coordinates within a space corresponding to the original data points within the training dataset.

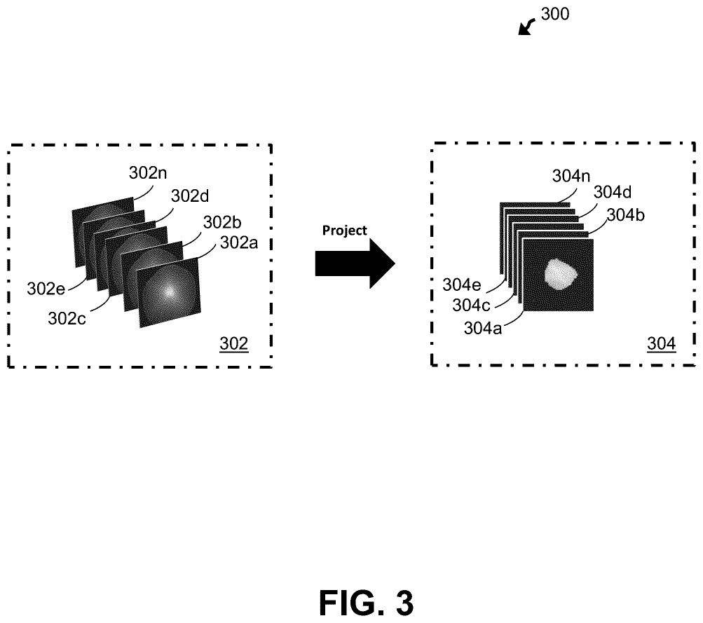

The analytics server may retrieve a DICOM RT dose file, a DICOM RT structure file, a DICOM CT file, and a DICOM RT plan file (block 208) and then construct a 3D dose tensor, a 3D structure tensor, and a 3D CT tensor (block 210) respectively. After preparing the tensors, the analytics server may project the 3D tensors onto 2D planes from the perspective of the beam's eye view. Each 2D projection may have a corresponding control point. For instance, the analytics server may generate a 2D projection based on the 3D tensor at each control point. Each 2D projection may also have a corresponding control weight at the corresponding control point. The 3D tensors and the 2D projections can be concatenated to form the input data used to train the AI model (e.g., deep learning model). For instance, the analytics server may generate the concatenated projected 2D dose and structure tensor (block 212) using the methods discussed herein. In a non-limiting example, as depicted in example 300 depicted in FIG. 3, the analytics server may receive (e.g., from a plan optimizer) the desired 3D dose planning structure.

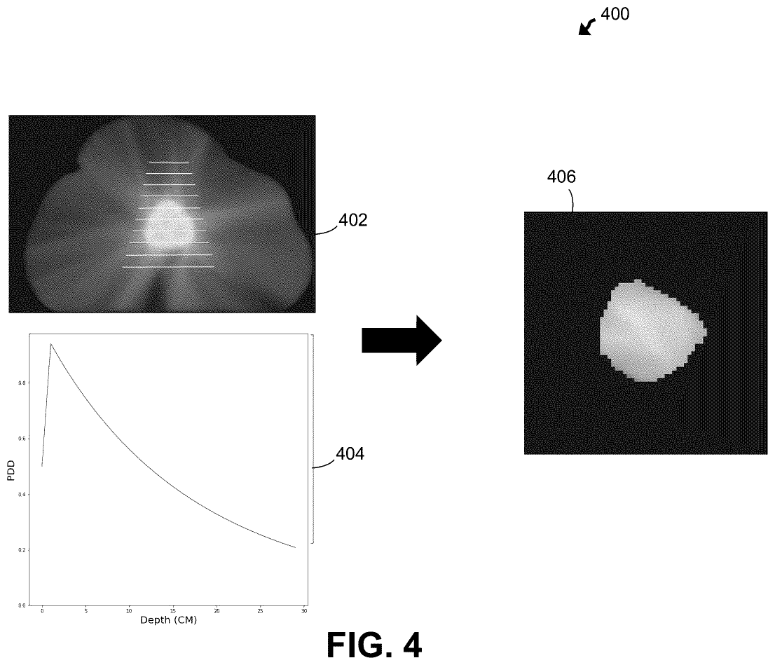

The desired 3D dose planning structure may be a sequence of dose planning structures 302. The analytics server may first desegregate the desired 3D dose planning structures 302 into frames 302a-n. The analytics server may then generate stacked 2D projections 304 that include time-stamped frames 304a-n. Each frame 304a-n corresponds to a plan of beam-eye view at a particular control point. Also, as depicted in FIG. 4 (example 400), for each desired 3D dose planning structure 402 (similar to the frames 302a-n), the analytics server may generate one projection per control point (projection 406). Each dose projection may also be generated/projected in accordance with a corresponding Percentage Depth Dose (PDD) curve. For instance, the projection 402 may be a PDD-based weighted average of each sampling plane based on the PDD 404. The projection 406 may also be a binary mask that corresponds to the planning structures.

Referring back to FIG. 2B, the analytics server may also analyze data associated with MLC sequence and control point weights (block 216) by extracting them from the DICOM RT plan files. The analytics server may then construct a tensor for numeric representation of MLC sequence from the extracted MLC sequence data (block 218). The analytics server may then generate a tensor for graphical representation of MLC sequence from the MLC sequence data retrieved and extracted (block 220). Moreover, the analytics server may then construct a tensor for control point weights from the control point weights retrieved and extracted (block 222).

Training the AI Model

Using the training dataset, the analytics server may train one or more AI models discussed herein. In various embodiments, the AI model may use one or more deep learning engines to perform automatic segmentation of images received and/or to correlate the data within the training dataset, such that they uncover patterns connecting how various prepared data corresponds to linear accelerator machine instructions (e.g., MLC openings and control point weights). Specifically, the AI model 224 may ingest data associated with blocks 212, 218, 220, and 222 to train itself.

The AI model may first analyze the prepared training dataset and determine a pattern among the tensors and MLC openings and control weights. Using various machine-learning techniques, the model may identify how each MLC opening and control weight corresponds to different RTTP attributes (filed, extracted, and prepared within the training dataset).

The AI model 224 may comprise a neural network comprising several layers of convolutional neural networks and may use a deep learning method to train itself. One type of deep learning engine is a deep neural network (DNN). A DNN is a branch of neural networks and consists of a stack of layers each performing a specific operation, e.g., convolution, pooling, loss calculation, etc. Each intermediate layer receives the output of the previous layer as its input. The beginning layer is an input layer, which is directly connected to or receives an input data structure that includes the data items in one or more machine-readable objects, and may have a number of neurons equal to the data items in one or more machine-readable objects provided as input. For example, a machine-readable object may be a data structure, such as a list or vector, which includes a number of data fields containing data within the training dataset. Each neuron in an input layer can accept the contents of one data field as input.

The next set of layers can include any type of layer that may be present in a DNN, such as a convolutional layer, a fully connected layer, a pooling layer, or an activation layer, among others. Some layers, such as convolutional neural network layers, may include one or more filters. The filters, commonly known as kernels, are of arbitrary sizes defined by designers. Each neuron can respond only to a specific area of the previous layer, called receptive field. The output of each convolution layer can be considered as an activation map, which highlights the effect of applying a specific filter on the input. Convolutional layers may be followed by activation layers to apply non-linearity to the outputs of each layer. The next layer can be a pooling layer that helps to reduce the dimensionality of the convolution layer's output. In various implementations, high-level abstractions are extracted by fully connected layers. The weights of neural connections and the kernels may be continuously optimized in the training phase.

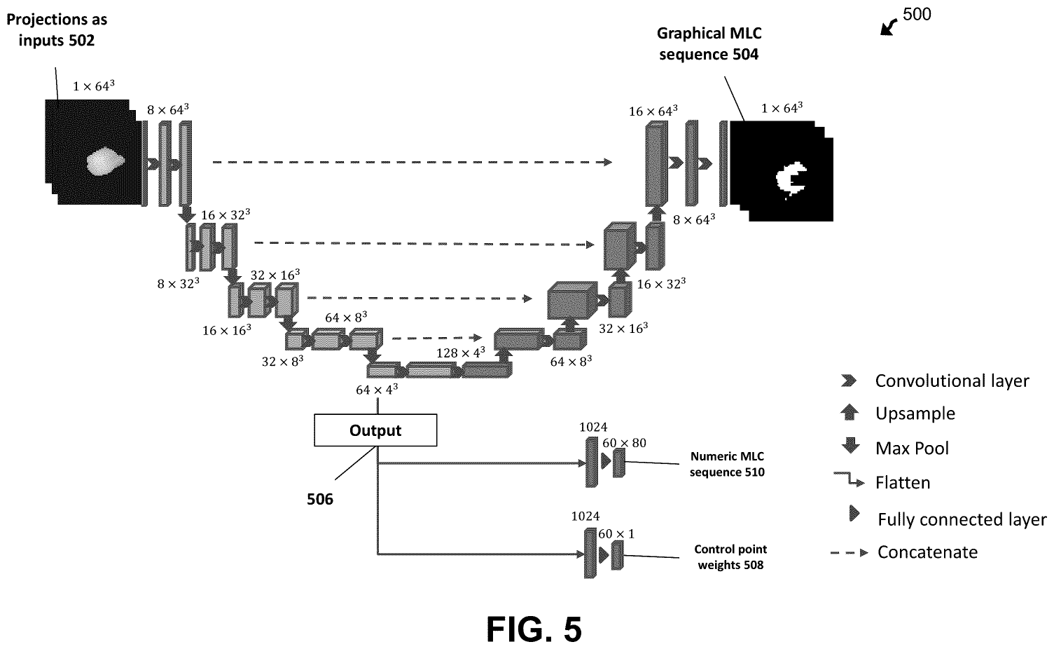

Referring now to FIG. 5, example 500 depicts how the AI model is trained using convolutional neural network-based multi-task learning framework. In the example 500, the AI model ingests dose and structure projections 502 and uses various encoding and decoding techniques to identify hidden patterns between the inputted data and a predicted output 506, such as graphical and/or numerical MLC sequence predictions 510 and/or control point weights 508.

In some embodiments, the AI model may be configured to use various machine learning techniques to generate a graphical representation of the MLC as well, such as the graphical MLC sequence 504. Using the predicted tensors, the AI model may also generate what an MLC opening should look like (e.g., a binary mask of the MLC opening).

Even though multi-task, supervised learning is discussed herein, other embodiments may include using other machine learning methods, such as reinforcement learning, adversarial learning, unsupervised learning, and the like. The training of the AI model may be performed using an unsupervised manner. In the unsupervised learning method, the relationship between RTTP attributes and the MLC opening and/or control weights may not always be known to the AI model (as opposed to supervised learning methods in which data points are labeled as the ground truth).

In some configurations, the analytics server may pre-train or partially train the AI model. For instance, the analytics server may train the AI model based on a set of cohort patients or clinic data. Then, the analytics server may train (fine-tune) the AI model using a particular patient's or a particular clinic's specific data. For instance, when the AI model is pre-trained, the analytics server may fine-tune the AI model and customize it to a particular patient (or a group of patients) by feeding information of the patient or a clinic. This allows for customizing the AI model without risking overfitting. For instance, because the number of possibilities is high when generating RTTP, each clinic or clinician may have their own preferences and may approach solving the same problem differently. As a result, the AI model may be customized for different users. Using data associated with a particular clinic (e.g., RTTPs and treatment data associated with patients who were treated at a particular clinic) allows the AI model to learn various attributes common among treatments administered to patients for that clinic. As a result, the AI model may then fine-tune its learning (and as a result its predicted results) for a particular clinic.

During training, the analytics server may iteratively produce new predicted results (e.g., projections) based on the training dataset (e.g., for each patient and their corresponding data). If the predicted results do not match the real outcome, the analytics server continues the training unless and until the computer-generated recommendation satisfies one or more accuracy thresholds and is within acceptable ranges. For instance, the analytics server may segment the training dataset into three groups (i.e., training, validation, and test). The analytics server may train the AI model based on the first group (training). The analytics server may then execute the (at least partially) trained AI model to predict results for the second group of data (validation). The analytics server then verifies whether the prediction is correct. Using the above-described method, the analytics server may evaluate whether the AI model is properly trained. The analytics server may continuously train and improve the AI model using this method. The analytics server may then gauge the AI model's accuracy (e.g., area under the curve, precision, and recall) using the remaining data points within the training dataset (test).

Implementation of the AI Model

Referring now to FIG. 2C, a flow diagram depicting the execution and results of the AI model. As depicted, the AI model 224 (when trained properly) may, at implementation time, generate a tensor for numeric representation of MLC sequence 226, tensor for graphical representation of MLC sequence 228, and/or tensor for control point weights 230. The AI model may use a regression-based loss function and/or Dice-coefficient loss function to evaluate its outputs against a defined loss. For instance, the AI model 224 may use the regression-based loss function to generate the blocks 226 and 230. The AI model may use a Dice-coefficient-based loss function to predict the block 228. A non-limiting example of a loss function may be a difference between a predicted MLC opening (a tensor associated with the MLC opening) and an actual MLC opening (e.g., within the training dataset). Another loss function may be defined as restrictions of MLC openings. MLC opening restrictions may correspond to physical or software restrictions on how MLC openings can be achieved.

The two tensors related to the MLC sequence (blocks 226 and 228) may be combined together to generate a numeric MLC sequence (block 232). The analytics server may optionally generate an RTTP file (DICOM plan file 234) that includes the results predicted by the AI model 224. This file may include machine-readable instructions that can be used to populate a GUI and/or directly instruct a radiation therapy machine to change its configurations (e.g., open an MLC in accordance with the data predicted by the AI model 224).

Referring back to FIG. 2A, at 204, the analytics server may receive treatment objectives for a patient including at least a 3D dose volume (e.g., dose-volume objective for at least one structure of a patient to be treated via a radiation therapy machine). The analytics server may receive treatment objectives from a clinician. Using various methods, the analytics server may generate or retrieve desired dose planning for different structures of the patient. For instance, the analytics server may instruct a plan optimizer to generate a 3D dose planning structure (e.g., DVHs) for the patient.

The input to the AI model (e.g., 3D dose volume/distribution) can be generated in multiple ways. It could be generated using MLC-position free optimization process based on using DVH objectives as inputs, or it could also be generated with another AI algorithm that predicts a 3D dose volume/distribution.

At step 206, the analytics server may execute an artificial intelligence model to predict an MLC opening position (e.g., numerical representation and/or an image or a binary mask of the MLC opening) and a corresponding movement attribute of an accelerator of the radiation therapy machine, wherein the artificial intelligence model is trained via a training dataset that comprises training treatment objectives and attributes associated with previously performed radiation therapy treatments comprising at least actual or projected 3D dose volumes for the treated patients and corresponding MLC opening images.

The analytics server may execute the AI model that has been trained using the methods discussed herein. The AI model may use the methods discussed herein to generate a tensor for numeric representation of MLC sequence, a tensor for graphical representation of MLC sequence, and/or a tensor for control point weights. The AI model may first analyze the received 3D dose volume and project MLC opening and control point weights accordingly.

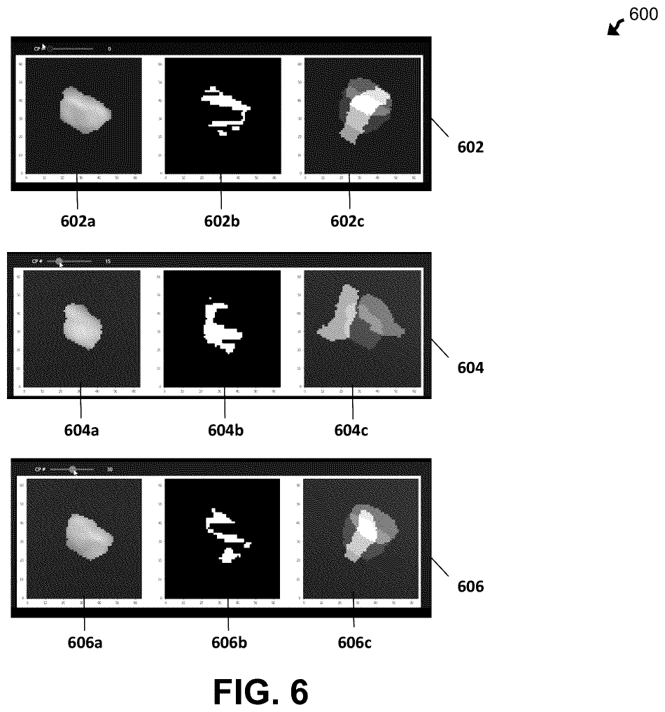

At step 208, the analytics server may present a predicted MLC opening position and a corresponding predicted movement attribute of the accelerator of the radiation therapy machine. The analytics server may populate a GUI using the results predicted by the AI model. Referring now to FIG. 6, a non-limiting example of a GUI presented by the analytics server is presented. The GUI 600 presents a moving image of the predicted MLC opening sequence during the patient's treatment. Therefore, the GUI 600 includes different frames (602, 604, and 606). Each frame corresponds to a particular timeframe or time of treatment for the patient. For instance, frame 602 corresponds to a 0 second timestamp (at the beginning of the treatment), frame 604 corresponds to 15^{th} second of the treatment, and frame 606 corresponds to 30^{th} second of the treatment.

Each frame may include a projected beam eye view 602a, 604a, and 606a. Each project beam eye view depicts a projected 2D MLC opening from the beam eye view, as discussed herein, e.g., in FIGS. 3-4. Each frame may also include a graphical representation of an MLC opening 602b, 604b, and 606b. These MLC openings are predicted by the AI model and represented by a binary mask associated with the opening itself, which is generated in accordance with MLC tensors (graphical and/or numerical) generated by the AI model. The frames may optionally include the graphical elements 602c, 604c, and 606c depicting a graphical representation (3D) of the structure receiving radiation.

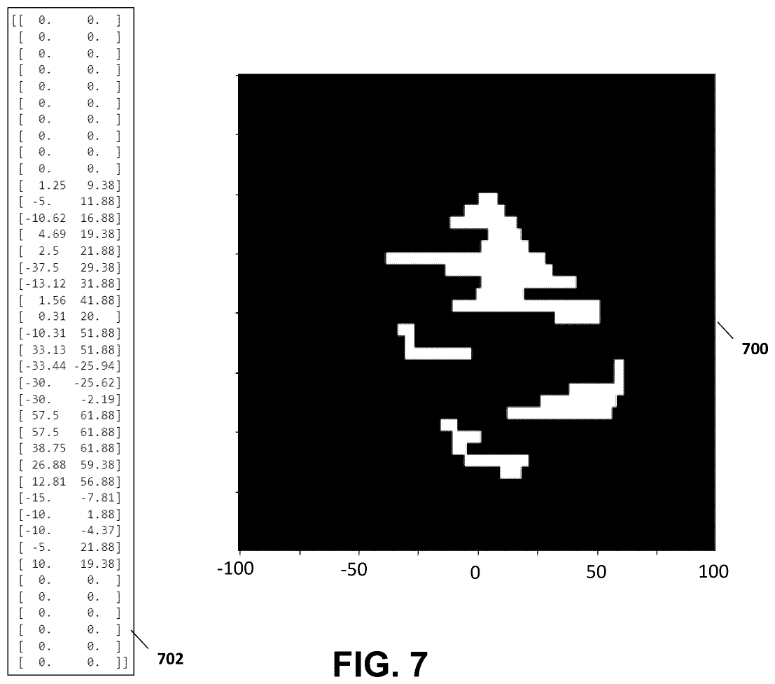

Referring now to FIG. 7, a numerical representation of the control point weights is depicted.

The numeric representation for control point weights may be a list of numbers. A full arc in VMAT may be defined by 178 equidistant control points to describe the machine rotation motion that delivers this arc. Therefore, the weights can be represented by a list of 178 numbers that add up to 1.0: the first number is typically the weight for the first control point, the second number is the weight for the second control point, etc. For instance, the numeric representation of the control points may be the following:
[0.0000, 0.0027, 0.0053, 0.0054, 0.0057, . . . , 0.0051, 0.0025]

In FIG. 7, the numerical representation 702 depicts numeric representation of 40 pairs of MLCs from a particular control point. For each pair, the first number is the position of the leaf edge of the left MLC and the second number is the position of the leaf edge of the right MLC.

Moreover, the binary mask 700 may depict a graphical representation of the same MLCs from the same control point (e.g., white=open, black=closed) as numerically represented via the numeral representation 702. For example, the first 10 pairs depicted all have 0's for each MLC, meaning that, at each row, the MLCs are in contact of each other at position 0 and therefore these MLCs are considered “closed” (e.g., black in the binary mask). For the 11^{th} pair (e.g., control points), the left MLC ends at position 1.25 mm, and the right MLC ends at position 9.38 mm, leaving a ˜8 mm gap in the middle, which is this part in the binary mask 700.

In another example, the analytics server may revise one or more attributes of the patient's radiation therapy treatment using the data predicted by the AI model. For instance, the analytics server may revise an attribute of the MLC (e.g., the MLC opening), move the treatment table (couch), pause the beam, or a combination of any of these examples using the control weights or predicted MLC data. Specifically, in conjunction with one or more other software solutions, the analytics server may revise an opening of the MLC, such that radiation dissemination is directed towards the projected location of a PTV (e.g., using an MLC opening attribute and/or control point weight that is projected using the AI model). In this way, the analytics server provides a dynamic MLC correction method where the MLC opening can be revised in real-time or near real-time. Effectively, the analytics server may enable gating of the beam to match the treatment objectives.

In another example, the analytics server may transmit the data predicted via the AI model to a downstream software solution. For instance, the results of the execution of the AI model can be transmitted to a dose calculation software solution, such as a plan optimizer. The plan optimizer may further analyze the RTTP using the data predicted via the AI model.

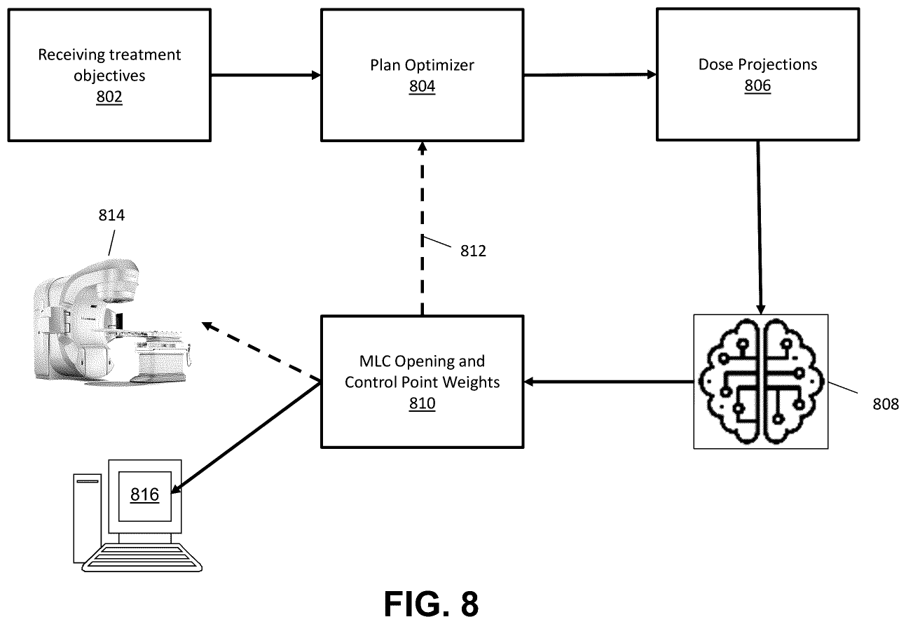

In a non-limiting example, such as example 800 depicted in FIG. 8, the analytics server may receive treatment objectives 802 from a treating physician. The analytics server may execute a plan optimizer 804 to generate various dose predictions 806 for the patient. The analytics server may then use the dose projections 806 to execute the AI model 808 to generate predicted MLC openings and control point weights 810. The analytics server may optionally transmit the predicted MLC openings and control point weights 810 back to the plan optimizer 804, such that the plan optimizer 804 can generate a revised RTTP for the patient from the data predicted by the AI model 808 (step 812). Moreover, the analytics server may optionally instruct the radiation therapy machine 814 to dynamically revise one or more of its configurations (e.g., MLC openings) in accordance with the results predicted by the AI model 808. Moreover, the analytics sever may present the results of execution of the AI model 808 on a user computing device 816.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What We claim is:

1. A method comprising:
receiving, by a processor, treatment objectives for a patient including at least a dose-volume for at least one structure of the patient to be treated via a radiation therapy machine;
executing, by the processor, an artificial intelligence model to predict an attribute of a Multi-Leaf Collimator (MLC) opening and a corresponding movement attribute of an accelerator of the radiation therapy machine,
wherein the artificial intelligence model is trained via a training dataset that comprises training treatment objectives and attributes associated with previously performed radiation therapy treatments comprising at least actual or projected dose-volume for the treated patients and corresponding MLC opening positions; and
presenting, by the processor, a predicted MLC opening position and a corresponding predicted movement attribute of the accelerator of the radiation therapy machine.

2. The method of claim 1, wherein the artificial intelligence model is trained via a deep learning protocol to correlate the actual or projected dose-volume for the treated patients and corresponding MLC opening position.

3. The method of claim 1, wherein the predicted MLC opening position is a binary mask indicating opening of the MLC.

4. The method of claim 1, wherein the predicted movement attribute is a time associated with the accelerator's movement.

5. The method of claim 1, wherein the predicted movement attribute is an angle associated with the accelerator's movement.

6. The method of claim 1, wherein the artificial intelligence model further predicts a sequence of MLC openings for the patient.

7. The method of claim 1, wherein the artificial intelligence model utilizes a loss function in accordance with MLC opening restrictions.

8. The method of claim 1, further comprising:
generating, by the processor, machine-readable instructions in accordance with the predicted MLC openings and corresponding predicted movement attributes.

9. The method of claim 8, further comprising:
transmitting, by the processor, the machine-readable instructions to the radiation therapy machine.

10. A computer system comprising:
a server comprising a processor and a non-transitory computer-readable medium containing instructions that when executed by the processor causes the processor to perform operations comprising:
receiving treatment objectives for a patient including at least a dose-volume for at least one structure of the patient to be treated via a radiation therapy machine;
executing an artificial intelligence model to predict an attribute of a Multi-Leaf Collimator (MLC) opening and a corresponding movement attribute of an accelerator of the radiation therapy machine,
wherein the artificial intelligence model is trained via a training dataset that comprises training treatment objectives and attributes associated with previously performed radiation therapy treatments comprising at least actual or projected dose-volume for the treated patients and corresponding MLC opening positions; and
presenting a predicted MLC opening position and a corresponding predicted movement attribute of the accelerator of the radiation therapy machine.

11. The computer system of claim 10, wherein the artificial intelligence model is trained via a deep learning protocol to correlate the actual or projected dose-volume for the treated patients and corresponding MLC opening position.

12. The computer system of claim 10, wherein the predicted MLC opening position is a binary mask indicating opening of the MLC.

13. The computer system of claim 10, wherein the predicted movement attribute is a time associated with the accelerator's movement.

14. The computer system of claim 10, wherein the predicted movement attribute is an angle associated with the accelerator's movement.

15. The computer system of claim 10, wherein the artificial intelligence model further predicts a sequence of MLC openings for the patient.

16. The computer system of claim 10, wherein the artificial intelligence model utilizes a loss function in accordance with MLC opening restrictions.

17. The computer system of claim 10, wherein the instructions further cause the processor to generate machine-readable instructions in accordance with the predicted MLC openings and corresponding predicted movement attributes.

18. The computer system of claim 17, wherein the instructions further cause the processor to transmit the machine-readable instructions to the radiation therapy machine.

19. A system comprising a server having one or more processors configured to:
receive treatment objectives for a patient including at least a dose-volume for at least one structure of the patient to be treated via a radiation therapy machine;
execute an artificial intelligence model to predict an attribute of a Multi-Leaf Collimator (MLC) opening and a corresponding movement attribute of an accelerator of the radiation therapy machine,
wherein the artificial intelligence model is trained via a training dataset that comprises training treatment objectives and attributes associated with previously performed radiation therapy treatments comprising at least actual or projected dose-volume for the treated patients and corresponding MLC opening positions; and
present a predicted MLC opening position and a corresponding predicted movement attribute of the accelerator of the radiation therapy machine.

20. The computer system of claim 19, wherein the artificial intelligence model is trained via a deep learning protocol to correlate the actual or projected dose-volume for the treated patients and corresponding MLC opening position.
